(19) United States
(12) Patent Application Publication
(10) Pub. No.: US 2019/0053841 A1
(43) Pub. Date: Feb. 21, 2019

(54) BONE HEMOSTAT AND CARRIER

(71) Applicant: WNDM Medical Inc., Fort Worth, TX (US)

(72) Inventors: Sherlene F. Bagley, Arlington, TX (US); Robert K. Mart, Colleyville, TX (US)

(21) Appl. No.: 16/104,837

(22) Filed: Aug. 17, 2018

Related U.S. Application Data
(60) Provisional application No. 62/547,675, filed on Aug. 18, 2017.

(51) Int. Cl. A61B 17/88

(52) U.S. Cl. CPC A61B 17/8811; A61B 2017/561

(57) ABSTRACT

The present invention includes compositions and methods for treating bone injury and surgical bone incisions and openings that include a bone hemostat material and tape delivery system comprising: one or more dots, beads, strips, tapes or ribbon-shaped beads of bone hemostatic material provided on a tray capable of being sterilized.

### CROSS-REFERENCE TO RELATED APPLICATIONS

This application claims priority to U.S. Provisional Patent Application No. 62/547,675, filed Aug. 18, 2017, entitled “Bone Wax Hemostat Tape Delivery System,” the entire contents of which are hereby fully incorporated herein by reference for all purposes.

### STATEMENT OF FEDERALLY FUNDED RESEARCH

Not applicable.

### TECHNICAL FIELD OF THE INVENTION

The present disclosure relates generally to hemostats and, more specifically, to bone hemostats and delivery systems.

### BACKGROUND OF THE INVENTION

Without limiting the scope of the invention, its background is described in connection with surgical procedures of bone.

Where a surgical procedure requires a surgeon to cut compact or cancellous bone to gain access to an anatomical cavity, for example, the sternum is a cancellous (open cell, cavernous) bone which is incised as part of a Coronary Artery Bypass Graft (CABG) surgical procedure, an unmet need exists to safely and immediately minimize blood flow from the incised bone during the procedure.

The present disclosure, therefore, provides novel and non-obvious solutions for controlling blood flow from an incised bone.

### SUMMARY OF THE INVENTION

In one embodiment, the present invention includes a bone hemostat material and tape delivery system comprising: one or more dots, beads, strips, tapes or ribbon-shaped beads of bone hemostatic material provided on a release carrier or tray capable of being sterilized. In one aspect, the bone hemostat material and tape delivery system further comprises a backing on the tape or ribbon-shaped bead of bone hemostatic material that is equal in width or wider than a width of the tape or ribbon-shaped bead of bone hemostatic material. In another aspect, the one or more beads, strips, tapes or ribbon-shaped beads of bone hemostatic material is provided in one or more strips on a biocompatible backing. In another aspect, the one or more beads, strips, tapes or ribbon-shaped beads of bone hemostatic material is 1-5 mm in depth, 5-40 mm in width, or 10-150 mm in length. In another aspect, the tape or ribbon-shaped bead of bone hemostatic material strip 1-5 mm in depth, 5-40 mm in width, and 10-150 mm in length. In another aspect, each of the one or more dots, beads, strips, tapes or ribbon-shaped beads of bone hemostatic material is packaged for single, individual sterilization and use. In another aspect, the one or more dots, beads, strips, tapes or ribbon-shaped beads of bone hemostatic material, a backing, or both comprise an adhesive. In another aspect, the one or more dots, beads, strips, tapes or ribbon-shaped beads of bone hemostatic material adheres to a bone without an adhesive. In another aspect, at least one edge of the tray can be used as a guide for the application of the one or more dots, beads, strips, tapes or ribbon-shaped beads of bone hemostatic material. In another aspect, the tray is flexible and provides a groove or indentation for the one or more dots, beads, strips, tapes or ribbon-shaped beads of bone hemostatic material and further comprises a backing for the direct application of the one or more dots, beads, strips, tapes or ribbon-shaped beads of bone hemostatic material to a site in need of treatment. In another aspect, the one or more dots or beads are shaped to equal or exceed the width of an opening in a drilled or reamed bone. In another aspect, the system further comprises a hand-held applicator stick or instrument with an end that is shaped for the application of the one or more dots, beads, strips, tapes or ribbon-shaped beads of bone hemostatic material. In another aspect, the stick or applicator would be pre-loaded with a small ball or sphere of the bone hemostat material. In another aspect, the one or more dots, beads, strips, tapes or ribbon-shaped beads of bone hemostatic material are resorbable. In another aspect, the stick or applicator is metal or plastic, is 10 cm to 40 cm (approximately 4″ to 12″) in length, inclusive of the hemostat material on one end, therein the hemostat material on the end of each applicator ranges from 3 mm to 12 mm diameter in the shape of a sphere or oval.

In another embodiment, the present invention includes a method of treating a bone in need of hemostasis comprising: providing a bone hemostat material and tape delivery system comprising: one or more dots, beads, strips, tapes or ribbon-shaped beads of bone hemostatic material provided on a tray capable of being sterilized. In one aspect, the method further comprises a backing on the tape or ribbon-shaped bead of bone hemostatic material that is equal in width or wider than a width of the tape or ribbon-shaped bead of bone hemostatic material. In another aspect, the one or more beads, strips, tapes or ribbon-shaped beads of bone hemostatic material is 1-5 mm in depth, 5-40 mm in width, or 10-150 mm in length. In another aspect, the one or more beads, strips, tapes or ribbon-shaped beads of bone hemostatic material is 10-150 mm in length. In another aspect, the tape or ribbon-shaped bead of bone hemostatic material strip 1-5 mm in depth, 5-40 mm in width, and 10-150 mm in length. In another aspect, each of the one or more dots, beads, strips, tapes or ribbon-shaped beads of bone hemostatic material is packaged for single, individual sterilization and use. In another aspect, the one or more dots, beads, strips, tapes or ribbon-shaped beads of bone hemostatic material, a backing, or both comprise an adhesive. In another aspect, the one or more dots, beads, strips, tapes or ribbon-shaped beads of bone hemostatic material adheres to a bone without an adhesive. In another aspect, at least one edge of the tray can be used as a guide for the application of the one or more dots, beads, strips, tapes or ribbon-shaped beads of bone hemostatic material. In another aspect, the tray is flexible and provides a groove or indentation for the one or more dots, beads, strips, tapes or ribbon-shaped beads of bone hemostatic material and further comprises a backing for the direct application of the one or more dots, beads, strips, tapes or ribbon-shaped beads of bone hemostatic material to a site in need of treatment. In another aspect, the one or more dots or beads are shaped to equal or exceed the width of an opening in a drilled or reamed bone. In another aspect, the method further comprises a hand-held applicator stick or instrument with an end that is shaped for the application of the one or more dots, beads, strips, tapes or ribbon-shaped beads of bone hemostatic material. In another aspect, the stick or applicator would be pre-loaded with a small ball or sphere of the bone hemostat material. In another aspect, the one or more dots, beads, strips, tapes or ribbon-shaped beads of bone hemostatic material are resorbable. In another aspect, the stick or applicator is metal or plastic, is 10 cm to 40 cm (approximately 4″ to 12″) in length, inclusive of the hemostat material on one end, therein the hemostat material on the end of each applicator ranges from 3 mm to 12 mm diameter in the shape of a sphere or oval.

### BRIEF DESCRIPTION OF THE DRAWINGS

For a more complete understanding of the features and advantages of the present invention, reference is now made to the detailed description of the invention along with the accompanying figures and in which:

FIG. 1 is a depiction of a bone hemostat and carrier, according to certain example embodiments;

FIG. 2 is a depiction of an incised sternum and application of a bone hemostat using a carrier, according to certain example embodiments;

FIG. 3 is a depiction of a specialized applicator for the bone hemostat material, according to certain example embodiments, and

FIGS. 4A-4C are a first and second, third depiction of containers used for storing and transporting the bone hemostats and carriers, according to certain example embodiments.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The present invention is a bone hemostat material and tape delivery system comprising: one or more dots, beads, strips, tapes or ribbon-shaped beads of bone hemostatic material provided on a tray capable of being sterilized. The bone hemostat material and tape delivery system further comprises a backing on the tape or ribbon-shaped bead of bone hemostatic material that is equal is width or wider than a width of the tape or ribbon-shaped bead of bone hemostatic material. The one or more beads, strips, tapes or ribbon-shaped beads of bone hemostatic material is provided in one or more strips on a biocompatible backing. In another aspect, the one or more beads, strips, tapes or ribbon-shaped beads of bone hemostatic material is 1-5 mm in depth, 5-40 mm in width, or 10-150 mm in length. The tape or ribbon-shaped bead of bone hemostatic material strip 1-5 mm in depth, 5-40 mm in width, and 10-150 mm in length. Each of the one or more dots, beads, strips, tapes or ribbon-shaped beads of bone hemostatic material is packaged for single, individual sterilization and use. The one or more dots, beads, strips, tapes or ribbon-shaped beads of bone hemostatic material, a backing, or both comprise an adhesive. In another aspect, the one or more dots, beads, strips, tapes or ribbon-shaped beads of bone hemostatic material adheres to a bone without an adhesive. In certain examples, at least one edge of the tray can be used as a guide for the application of the one or more dots, beads, strips, tapes or ribbon-shaped beads of bone hemostatic material. The tray can be flexible and include a groove or indentation for the one or more dots, beads, strips, tapes or ribbon-shaped beads of bone hemostatic material and further comprises a backing for the direct application of the one or more dots, beads, strips, tapes or ribbon-shaped beads of bone hemostatic material to a site in need of treatment. The one or more dots or beads can be shaped to equal or exceed the width of an opening in a drilled or reamed bone. The system may further comprise a hand-held applicator stick or instrument with an end that is shaped for the application of the one or more dots, beads, strips, tapes or ribbon-shaped beads of bone hemostatic material. The invention can also include a stick or applicator would be pre-loaded with a small ball or sphere of the bone hemostat material. The one or more dots, beads, strips, tapes or ribbon-shaped beads of bone hemostatic material are resorbable. The stick or applicator is metal or plastic, is 10 cm to 40 cm (approximately 4″ to 12″) in length, inclusive of the hemostat material on one end, therein the hemostat material on the end of each applicator ranges from 3 mm to 12 mm diameter in the shape of a sphere or oval.

The present invention also includes a method of treating a bone in need of hemostasis comprising: providing a bone hemostat material and tape delivery system comprising: one or more dots, beads, strips, tapes or ribbon-shaped beads of bone hemostatic material provided on a tray capable of being sterilized. The method may further comprise adding a backing on the tape or ribbon-shaped bead of bone hemostatic material that is equal is width or wider than a width of the tape or ribbon-shaped bead of bone hemostatic material.

The example embodiments presented herein are directed to a bone hemostat for use in surgical operations to assist in controlling blood loss from an incised bone. For example, the sternum is an open cell, cancellous bone, which is incised as part of a Coronary Artery Bypass Graft (CABG) surgical procedure. The bone hemostat and storage and transport tray presented herein provide an effective and sterile way in which blood loss is minimized during these types of surgical procedures and ways to effectively store and transport bone hemostats.

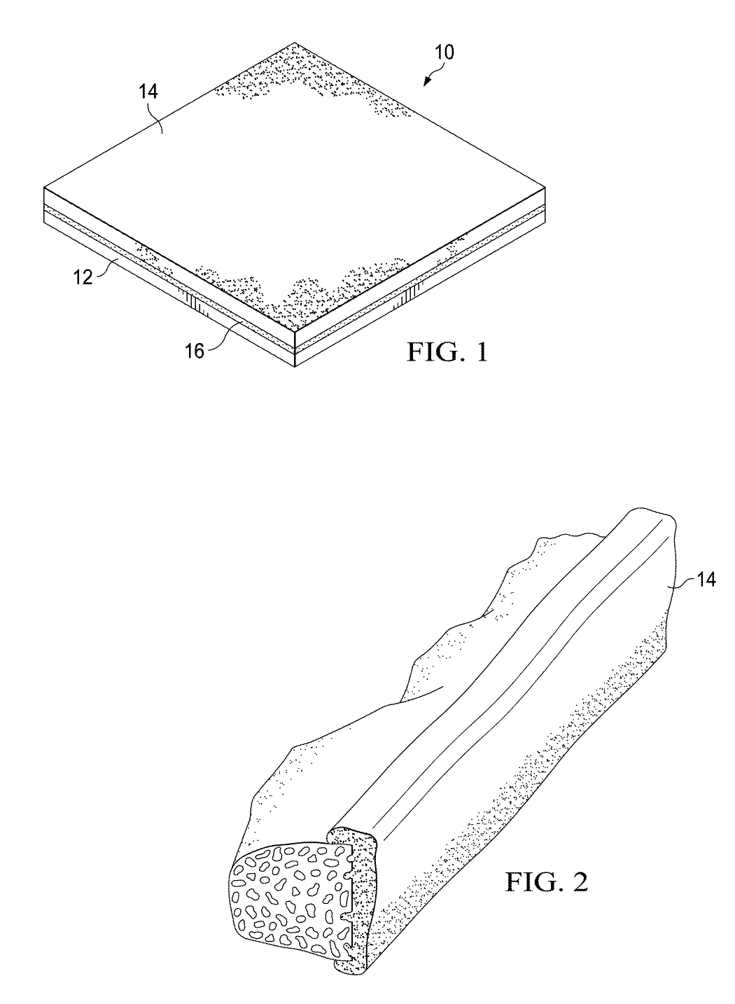

Referring now to FIG. 1, illustrated is a bone hemostat and carrier, according to certain example embodiments, denoted generally as 10. The hemostat and carrier 10 includes a carrier section 12 and a bone hemostat 14. The carrier section 12 can be a polymer-based carrier that can be made from a non-gel, bioerodable or non-biodegradable, biocompatible, occlusive or semi-occlusive polymer or paper coated with a suitable polymer or release layer. The carrier section 12 can optionally include an adhesive layer 16 in communication with the bone hemostat 14. The bone hemostat 14 can be a resorbable or non-resorbable hemostat made from a putty-like polymer. In an example, the bone hemostat 14 can be Signafuse Bioactive Bone Graft Putty available from Resorbable Orthopedic Products, LLC or Fort Worth, Tex., or other suitable materials. The putty-like polymer can be of a uniform thickness, width, and length. In addition, the bone hemostat and carrier 10 can be sterile.

The bone hemostat 14, either resorbable or non-resorbable, can be affixed to an incised bone, see FIG. 2, using the polymer carrier 12. A health care provider can deliver and secure the bone hemostat 14 to a section of incised bone by using the carrier 12. Once the absorbent layer 12 is secured, if the carrier 12 is not biodegradable, the backing can be removed. However, if the carrier 12 is biodegradable, it can remain in place, as illustrated in FIG. 2. During operation and post operations, the bone hemostat remains in-situ controlling blood flow.

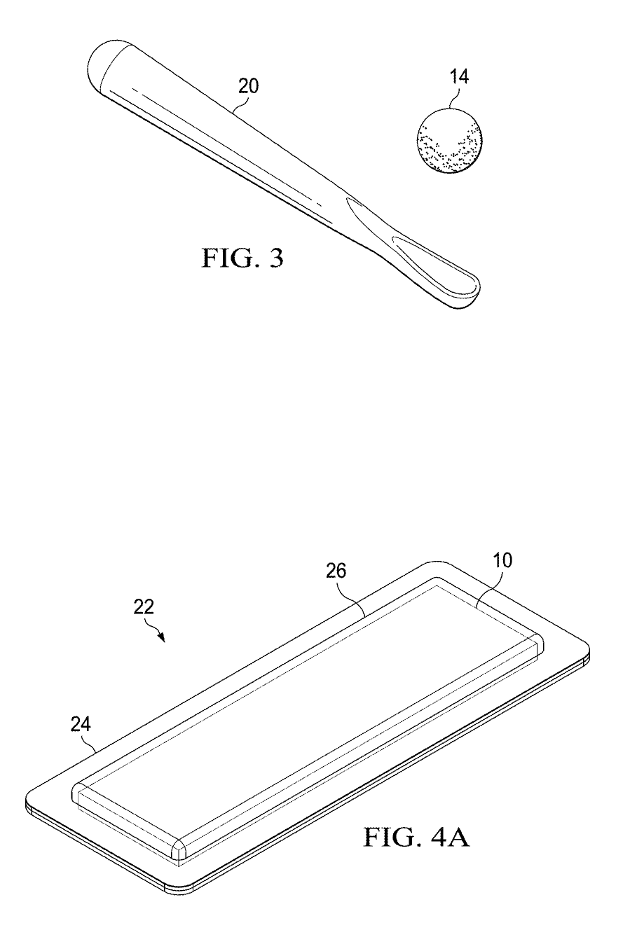

Referring now to FIG. 3, illustrated is a specialized bone hemostat applicator 20, according to certain example embodiments, for applying bone hemostat 14 to drilled or reamed bone. The applicator 20 includes a handle, tubular e.g., and a section defined by an opening into a cavity that can be used to scoop bone hemostat 14 from the hemostat and carrier 10, e.g., or pre-manufactured bone hemostat spheres can be provided. The applicator 20 can be a single, integrated piece, e.g. formed using plastic injection molding techniques or made from metal acceptable for use in surgical procedures. The handle can also include ribs, texture, a roughened surface or other suitable surface treatments or coverings to improve the ability of the applicator 20 to be held by a surgeon or other suitable medical personal during a medical procedure. The individual spheres of bone hemostat 14 can be placed onto the carrier section 12, with or without an adhesive layer 16. The dots are designed for easy application to drilled or reamed bone, for example, pilot holes for bone screws or intramedullary fixation. The dots can range in size for 3 mm to 12 mm.

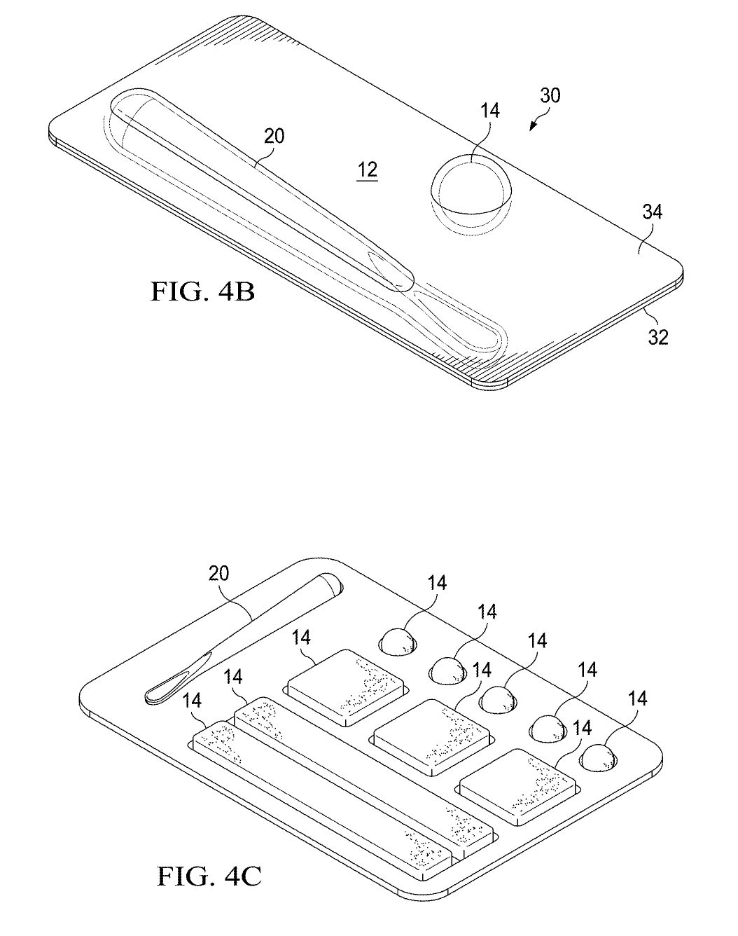

Referring now to FIGS. 4A-4C, illustrated are example embodiments of tray and containers for storing and transporting the bone hemostats and carriers.

FIG. 4A, illustrates one embodiment of a container 22 for transporting and storing carrier and bone hemostat 10. The container 22 includes a tray 24 for receiving a single strip of the bone hemostat and carrier 10 and a lid 26 for covering the hemostat 14. The tray 24 can be made from molded polymer using injection molding processes or other suitable materials and methods. As an example, the tray 24 can be the size of a single carrier and hemostat or about 10 1-5 mm in depth, 5-40 mm in width, and 10-150 mm in length. The bone hemostat 14 can be placed in the tray 24 and the carrier 12 applied on top of the bone hemostat 14.

The lid 26 can then be placed over the carrier 12. Alternatively, the bone hemostat 14 can be applied to the carrier 12 and placed in the tray 24 with the bone hemostat 14 protruding from the top. The lid 26 can then be placed over the section of bone hemostat 14 for transport. The lid 26 when positioned over the bone hemostat 14 and coupled with the tray 24 can be made to be flush with the surface of the tray 24. During application of the hemostat 14, the lid 24 can be removed and the tray 24 can be used to guide application of the hemostat 14 to incised bone. Obviously, the hemostat and carrier 10 can be removed from the tray 24 and used to apply the hemostat 14 to incised bone. Additionally, the tray 24 can include the hemostat 14 itself without the carrier 12. In other words, the tray 24 can function as the carrier 12 and the guide.

FIG. 4B illustrates another embodiment of a container 30 for transporting and storing bone hemostat 14 and applicator 20. The container 30 includes a tray 32 and a lid 34. The tray 32 can include carrier 12 that comprises a flat section or the carrier 12 can have pre-formed wells into which bone hemostat spheres can be placed for secure transport. The carrier 12 can comprise a thin flexible material that allows the bone hemostat 14 to be easily pressed into the pre-formed well. The number of bone hemostat dots on carrier 12 can vary from one to twenty, e.g., depending on the surgical procedure and number of holes required. In other words, trays can be designed for particular surgical procedures.

FIG. 4C illustrates yet another embodiment of container 40 for transporting and storing carrier and bone hemostat 10. The container can be of depth, length, and width to accommodate plurality of the carrier and bone hemostat 10. The bone hemostat 14 can be placed in the tray and then covered with the biodegradable or non-biodegradable backing used to deliver the hemostat to the wound. Positioning the hemostat and carrier in the tray in this manner allows for the product to be stored and transported without compromising the integrity of the product.

A system is also provided herein that includes a bone hemostat and carrier system for applying the bone hemostat to an incised bone in order to control the flow of bleeding during a surgical procedure. The bone hemostat can be resorbable or non-resorbable and the carrier system can be bioerodable or biocompatible. Depending on the characteristics, the system can remain in-situ post operation.

In one aspect, a bone hemostat system is provided that includes a carrier strip and a bone hemostat applied to a surface of the carrier strip. The carrier strip can be used to guide the bone hemostat to an incised bone so that the bone hemostat can be applied thereto. The carrier strip comprises one of a non-gel, bioerodable, non-bioerodable, biocompatible, occlusive, or semi-occlusive back support section. The bone hemostat can be resorbable or non-resorbable.

In another aspect, the bone hemostat system can also include a tray having a base section, a sidewall section surrounding and continuing the base section, and an interior section. A bone hemostat and carrier strip combination can be positioned in the tray, or alternatively only a bone hemostat can be positioned in the tray. A lid can be placed over the tray to protect the bone hemostat during storage and transport. The lid can be removed so as to expose a protruding member of the bone hemostat and the tray can be used to guide the bone hemostat to an incised bone.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A bone hemostat material and tape delivery system comprising:
one or more dots, beads, strips, tapes or ribbon-shaped beads of bone hemostatic material provided on a tray capable of being sterilized.

2. The material of claim 1, further comprising a backing on the tape or ribbon-shaped bead of bone hemostatic material that is equal is width or wider than a width of the tape or ribbon-shaped bead of bone hemostatic material.

3. The system of claim 1, wherein the one or more beads, strips, tapes or ribbon-shaped beads of bone hemostatic material is provided in one or more strips on a biocompatible backing.

4. The system of claim 1, wherein the one or more beads, strips, tapes or ribbon-shaped beads of bone hemostatic material is 1-5 mm in depth, 5-15 mm in width, or 10-150 mm in length.

5. The system of claim 1, wherein the tape or ribbon-shaped bead of bone hemostatic material strip 1-5 mm in depth, 5-15 mm in width, and 10-150 mm in length.

6. The system of claim 1, wherein each of the one or more dots, beads, strips, tapes or ribbon-shaped beads of bone hemostatic material is packaged for single, individual sterilization and use.

7. The system of claim 1, wherein the one or more dots, beads, strips, tapes or ribbon-shaped beads of bone hemostatic material, a backing, or both comprise an adhesive.

8. The system of claim 1, wherein the one or more dots, beads, strips, tapes or ribbon-shaped beads of bone hemostatic material adheres to a bone without an adhesive.

9. The system of claim 1, wherein at least one edge of the tray can be used as a guide for the application of the one or more dots, beads, strips, tapes or ribbon-shaped beads of bone hemostatic material.

10. The system of claim 1, wherein the tray is flexible and provides a groove or indentation for the one or more dots, beads, strips, tapes or ribbon-shaped beads of bone hemostatic material and further comprises a backing for the direct application of the one or more dots, beads, strips, tapes or ribbon-shaped beads of bone hemostatic material to a site in need of treatment.

11. The system of claim 1, wherein the one or more dots or beads are shaped to equal or exceed the width of an opening in a drilled or reamed bone.

12. The system of claim 1, further comprising a hand-held applicator stick or instrument with an end that is shaped for the application of the one or more dots, beads, strips, tapes or ribbon-shaped beads of bone hemostatic material.

13. The system of claim 12, wherein the stick or applicator would be pre-loaded with a small ball or sphere of the bone hemostat material.

14. The system of claim 1, wherein the one or more dots, beads, strips, tapes or ribbon-shaped beads of bone hemostatic material are resorbable.

15. The system of claim 12, wherein the stick or applicator is metal or plastic, is 4″ to 8″ in length, inclusive of the hemostat material on one end, therein the hemostat material on the end of each applicator ranges from 3 mm to 12 mm diameter in the shape of a sphere or oval.

16. A method of treating a bone in need of hemostasis comprising:
providing a bone hemostat material and tape delivery system comprising: one or more dots, beads, strips, tapes or ribbon-shaped beads of bone hemostatic material provided on a tray capable of being sterilized.

17. The method of claim 16, further comprising a backing on the tape or ribbon-shaped bead of bone hemostatic material that is equal is width or wider than a width of the tape or ribbon-shaped bead of bone hemostatic material.

18. The method of claim 16, wherein the one or more beads, strips, tapes or ribbon-shaped beads of bone hemostatic material is 1-5 mm in depth, 5-15 mm in width, or 10-150 mm in length.

19. The method of claim 16, wherein the one or more beads, strips, tapes or ribbon-shaped beads of bone hemostatic material is 10-150 mm in length.

20. The method of claim 16, wherein the tape or ribbon-shaped bead of bone hemostatic material strip 1-5 mm in depth, 5-15 mm in width, and 10-150 mm in length.

21. The method of claim 16, wherein each of the one or more dots, beads, strips, tapes or ribbon-shaped beads of bone hemostatic material is packaged for single, individual sterilization and use.

22. The method of claim 16, wherein the one or more dots, beads, strips, tapes or ribbon-shaped beads of bone hemostatic material, a backing, or both comprise an adhesive.

23. The method of claim 16, wherein the one or more dots, beads, strips, tapes or ribbon-shaped beads of bone hemostatic material adheres to a bone without an adhesive.

24. The method of claim 16, wherein at least one edge of the tray can be used as a guide for the application of the one or more dots, beads, strips, tapes or ribbon-shaped beads of bone hemostatic material.

25. The method of claim 16, wherein the tray is flexible and provides a groove or indentation for the one or more dots, beads, strips, tapes or ribbon-shaped beads of bone hemostatic material and further comprises a backing for the direct application of the one or more dots, beads, strips, tapes or ribbon-shaped beads of bone hemostatic material to a site in need of treatment.

26. The method of claim 16, wherein the one or more dots or beads are shaped to equal or exceed the width of an opening in a drilled or reamed bone.

27. The method of claim 16, further comprising a hand-held applicator stick or instrument with an end that is shaped for the application of the one or more dots, beads, strips, tapes or ribbon-shaped beads of bone hemostatic material.

28. The system of claim 27, wherein the stick or applicator would be pre-loaded with a small ball or sphere of the bone hemostat material.

29. The method of claim 16, wherein the one or more dots, beads, strips, tapes or ribbon-shaped beads of bone hemostatic material are resorbable.

30. The method of claim 16, wherein the stick or applicator is metal or plastic, is 4″ to 8″ in length, inclusive of the hemostat material on one end, therein the hemostat material on the end of each applicator ranges from 3 mm to 12 mm diameter in the shape of a sphere or oval.
